(19) United States
(12) Patent Application Publication
(10) Pub. No.: US 2024/0426734 A1
(43) Pub. Date: Dec. 26, 2024

(54) NOVEL MICROFLUIDIC CELL FABRICATION FOR ELECTRICAL ENHANCED OIL RECOVERY STUDIES

(71) Applicant: SAUDI ARABIAN OIL COMPANY, Dhahran (SA)

(72) Inventors: Dong Kyu Cha, Al-Shafa (SA); Ghaida Aljuhani, Dhahran (SA); Subhash C. Ayirala, Dhahran (SA); Abdulaziz S. Qasim, Dhahran (SA); Ali Abdallah Al-Yousef, Dhahran (SA)

(73) Assignee: SAUDI ARABIAN OIL COMPANY, Dhahran (SA)

(21) Appl. No.: 18/338,114

(22) Filed: Jun. 20, 2023

(51) Int. Cl. G01N 15/08; B01L 3/00; G01N 33/24

(52) U.S. Cl. CPC G01N 15/088; B01L 3/502715; G01N 33/241; B01L 2200/12; B01L 2300/0645; B01L 2300/168

(57) ABSTRACT

A device includes a microfluidic cell including a substrate and metal electrodes disposed on the substrate, a glass window disposed over the substrate, a current-voltage analyzer connected to the metal electrodes, and an inlet and an outlet in fluid communication with the microfluidic cell. A method of fabricating the device includes photolithographically exposing site for the metal electrodes utilizing a photoresist, depositing the metal electrodes on the substrate, and photolithographically patterning the substrate. A method of measuring pore throat size changes and oil mobilization includes connecting a current-voltage analyzer to a microfluidic cell and sweeping a current through the microfluidic cell via the current-voltage analyzer.

### BACKGROUND

After initial phases of oil recovery, enhanced oil recovery (EOR) methods may be used to increase production from mature wells. As global energy demands increase and more wells pass the initial phases of oil recovery, EOR methods are increasingly desirable because of a reduced use of water, energy, and chemicals. One efficient and environmentally preferred method of EOR is electrical EOR (EEOR). EEOR uses electricity to reduce oil viscosity by increasing the temperature. The varied application mechanisms of electricity allow for various EEOR methods. EEOR is one of the most promising EOR techniques because of its electrical efficiency and overall usefulness while minimizing environmental impact.

Rock pore permeability and pore throat structure are important factors involved in oil recovery, especially when a mature well undergoes EOR. To understand and improve aspects of EOR, it is desirable to study formation rock pore permeability and pore throat structure. Pores are voids in rock which can hold oil in an oil reservoir. The pore size and number will determine how much oil that reservoir can hold. The size and connectivity of the smaller passages connecting the pores, called pore throats, will affect how easily that oil can be recovered. A higher connectivity or increased size of pore throats would increase the productivity of a well. However, for EEOR, there remains a need for equipment for laboratory testing and characterization to estimate the impact of electrical current on pore throat size to effectively design the electric current parameters and the treatment time for EEOR applications. Thus, there is a need for a device to characterize enlarging and enhancing pore throat and permeability with electrical current.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a device which includes: a microfluidic cell including a substrate and metal electrodes disposed on the substrate; a glass window disposed over the substrate; a current-voltage analyzer connected to the metal electrodes; and an inlet and an outlet in fluid communication with the microfluidic cell.

In another aspect, embodiments disclosed herein relate to a method of fabricating a device, the device including a microfluidic cell which includes a substrate and metal electrodes disposed on the substrate; a glass window disposed over the substrate; a current-voltage analyzer connected to the metal electrodes; and an inlet and an outlet in communication with the microfluidic cell, the method including: photolithographically exposing site for the metal electrodes utilizing a photoresist; depositing the metal electrodes on the substrate; and photolithographically patterning the substrate.

In yet another aspect, embodiments disclosed herein relate to a method of measuring pore throat size changes and oil mobilization which includes: connecting a current-voltage analyzer to a microfluidic cell; and sweeping a current through the microfluidic cell via the current-voltage analyzer.

Other aspects and advantages of the claimed subject matter will be apparent from the following description and the appended claims.

### BRIEF DESCRIPTION OF DRAWINGS

FIG. 1 is a schematic of a device in accordance with one or more embodiments of the present disclosure.

FIG. 2 is a schematic of a device in accordance with one or more embodiments of the present disclosure.

FIG. 3 is a block-flow diagram of a method in accordance with one or more embodiments of the present disclosure.

FIG. 4 is a block-flow diagram of a method in accordance with one or more embodiments of the present disclosure.

FIG. 5 is a block-flow diagram of a method in accordance with one or more embodiments of the present disclosure.

### DETAILED DESCRIPTION

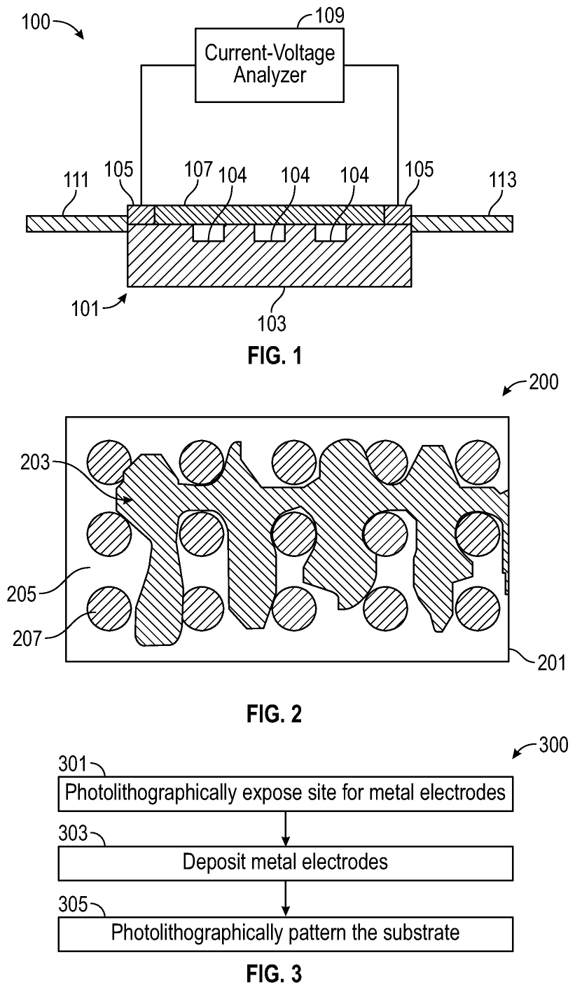

In one aspect, embodiments disclosed herein relate to a device for use in EOR studies, and especially in EEOR studies. In one or more embodiments of the present disclosure as shown in FIG. 1, the device 100 includes a microfluidic cell 101, which includes substrate 103, channels 104, metal electrodes 105. Device 100 further includes window 107, current-voltage analyzer 109, inlet 111, and outlet 113.

Microfluidic cell 101 is configured to probe the behavior of fluids at sub-millimeter levels, where surface forces dominate volumetric forces. This scale is useful for probing features of formation rock at that size. Device 100 can be used as a model to probe several important aspects of reservoir rock formations and allow for advanced measurements. For example, pore throat size distribution can be determined using pressure measurements with device 100 just as in a reservoir formation. This is useful as a formation's productivity is defined by the formation's pore throat characteristics. Device 100 can be used to probe and enhance productivity of mature reservoirs by optimizing performance in microfluidic laboratory studies. Microfluidic cell 101 can provide observations of changing pore throat size or oil mobilization. Microfluidic cell 101 allows for the determination of the optimal voltage and treatment time of electric current in EEOR studies by observing changing pore throat size and oil mobilization. In one or more embodiments, microfluidic cell 101 focuses on observation and characterization of enlarging pore throat size for enhancing permeability and oil mobilization when applying electrical current. By studying formation rock in device 100, the electrical conditions for EEOR can be optimized for field applications.

Microfluidic cell 101 includes a substrate 103. Suitable materials that may be present in the substrate 103 include silica. Suitable materials used for substrate 103 are desirably etchable and able to withstand the testing conditions in terms of temperature and chemical environment.

Substrate 103 includes channels 104 therein. Channels 104 may be sized suitably as known in the art such that channels 104 are configured to act as microfluidic channels. While shown in FIG. 1, with three channels, it will be understood that the number of channels may vary from three. Substrate 103 may be patterned with a suitable number of channels 104 for microfluidic cell 101 to operate. Channels 104 may be configured to mimic the structure of formation rock.

Microfluidic cell 101 includes metal electrodes 105 disposed on the substrate 103. Suitable metals that may be present in the metal electrodes 105 include gold, copper, titanium, silver, and platinum. Suitable materials allow metal electrodes 105 to be connection sites for electrical equipment. While two electrodes 105 are shown in FIG. 1 it will be understood that the number of electrodes may vary as suitable for the type of connection to the electrical equipment. Further, the metal electrodes 105 may be of the same or different materials and sizing from each other.

Microfluidic cell 101 is covered with a window 107. Suitable materials that may be present in the window include glass and transparent plastic. Glass is more preferred for higher temperature applications. Window 107 allows for microfluidic cell 101 to be part of a closed fluid system.

Metal electrodes 105 are connected to a current-voltage analyzer 109. Metal electrodes 105 are configured for application of electrical current to the microfluidic cell 101 from current-voltage analyzer 109. The current-voltage analyzer 109 allows for sweeping of electrical current to probe fluid properties. While illustrated in FIG. 1 with current-voltage analyzer 109, it will be understood that the metal electrodes 105 may be connected to other electrical equipment that would allow for the application of controlled current and voltage to microfluidic cell 101 via the metal electrodes 105.

The microfluidic cell 101 is connected to an inlet 111 and an outlet 113, allowing for fluid communication with the microfluidic cell. Such a connection of microfluidic cell 101 to inlet 111 and outlet 113 allows for pressure measurements using microfluidic cell 101. Further connecting fluid lines to the inlet 111 and outlet 113 allows measurement of fluid properties in the microfluidic cell, including pressure.

FIG. 2 shows a possible configuration of channels in the microfluidic cell, depicting a section from the interior of the microfluidic cell shown from an overhead view. A glass window 201 covers the top of the cell, underneath which brine 203 flows through channels 205 that are formed by the raised sections of the substrate 207 that were not removed during fabrication.

In another aspect, embodiments herein disclosed relate to a method of fabricating a device for use in EOR studies, and especially in EEOR studies. The device may be the device of FIG. 1. In one or more embodiments, a method 300 as shown in FIG. 3 for fabricating the device includes photolithographically exposing sites for metal electrodes, at block 301, depositing metal electrodes, at block 303, and photolithographically patterning the substrate, at block 305. The combination of photolithography and metal electrode deposition can result in fabrication of a microfluidic cell that can apply electrical current to study and optimize EEOR.

In one or more embodiments, photolithographically exposing sites for metal electrodes at block 301 includes applying a photoresist polymer coating to the substrate, then curing the photoresist coating. Curing the photoresist coating includes baking out the photoresist. As used herein, baking out the photoresist refers to curing the photoresist with heat. It will be understood that other suitable photoresist curing methods may be utilized. Suitable materials that may be present in the photoresist include polydimethylsiloxane (PDMS), polymethyl methacrylate (PMMA), and epoxy-based photoresists including SU-8. Suitable materials for the photoresist are coatable on the substrate and curable. Once the photoresist is cured, sections can be removed with ultraviolet lithography. Thus, after curing the photoresist, portions of the photoresist are removed using ultraviolet lithography to serve as sites for metal electrode deposition.

In one or more embodiments, depositing metal electrodes, at block 303 includes depositing metal with chemical vapor deposition so as to form the metal electrodes. Thus, after sites for metal electrode deposition are exposed on the substrate, chemical vapor deposition is used to deposit the metal electrodes on the exposed sites. In such embodiments, the combination of ultraviolet lithography and chemical vapor deposition allows for selective metal electrode placement.

In one or more embodiments, photolithographically patterning the substrate at block 305 includes exposing the photoresist and substrate to ultraviolet radiation with a mask to form a pattern in the photoresist. After patterning the photoresist with the mask, dry etching may be performed to remove substrate material from the areas not covered by photoresist. Removal of the substrate material leads to patterning of the substrate. The mask may have a structure based on a formation rock. Thus, the pattern may be representative of a formation rock. For example, the substrate may be patterned on the formation rock such that the pattern mimics the formation rock.

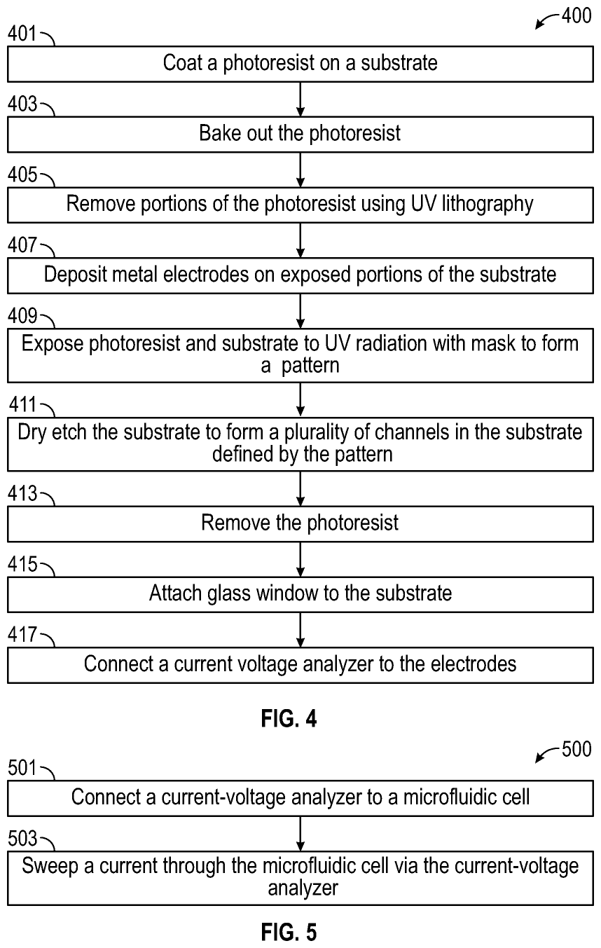

In one aspect, embodiments disclosed herein relate to a method 400 shown in FIG. 4 of fabricating the device of FIG. 1, illustrating implementation of method 300 shown in FIG. 3. This method 400 includes coating a photoresist on a substrate, at block 401, baking out the photoresist, at block 403, removing portions of the photoresist with ultraviolet lithography, at block 405, depositing metal electrodes on exposed portions of the substrate, at block 407, exposing the photoresist and substrate to ultraviolet radiation with a mask to form a pattern in the photoresist, at block 409, dry etching the substrate to form a plurality of channels in the substrate defined by the pattern in the photoresist, at block 411, removing the photoresist, at block 413, attaching a glass window to the substrate, at block 415, and connecting a current-voltage analyzer, at block 417.

In one or more embodiments, removing portions of the photoresist with ultraviolet lithography at block 403 includes removing selected portions of the substrate that are of sufficient size and suitable shape to serve as a deposition site for metal electrodes, typically 500 nm-50 μm in size and about 100 μm-500 μm apart. The removed portions correspond in design to a desired channel structure and in size to a channel depth.

In one or more embodiments, depositing the metal electrodes at block 407 is achieved using chemical vapor deposition. Suitable metals that may be present in the electrodes include gold, copper, titanium, silver, and platinum. In one or more embodiments, depositing the metal electrodes at block 407 includes depositing a pair of metal electrodes. It will be understood that another number of metal bodies with the potential of acting as electrodes is possible if their connection to electrical current results in electrical current in the cell.

In one or more embodiments, exposing the photoresist and substrate to ultraviolet radiation at block 409 includes using a mask to form a pattern in the photoresist. A pattern in the mask is transferred into the photoresist by ultraviolet radiation removing the portions of the photoresist that were not covered by the mask. When the mask used is modeled after formation rock, this step will transfer the structure of formation rock into the pattern of the photoresist.

In one or more embodiments, transferring the pattern of the photoresist that was defined by the mask to the substrate is achieved by dry etching the substrate at block 411, which forms a plurality of channels in the substrate defined by the pattern of the photoresist. The dry etching process only removes substrate material not covered by the photoresist which is the pattern imparted by the mask. When the mask used is modeled on formation rock, the etching process forms a plurality of channels in the substrate patterned on formation rock. After the pattern imparted by mask has been etched into the substrate the photoresist can be removed. The complete removal of the photoresist leaves behind a microfluidic cell with metal electrodes and a substrate with a plurality of channels patterned on formation rock.

In one or more embodiments, attaching a window to the substrate at block 415 forms a closed microfluidic cell. Suitable materials that may be present in the window include glass. The window allows for viewing of the microfluidic cell. In one or more embodiments, a current-voltage analyzer is attached to the metal electrodes at block 417. Attaching the current-voltage analyzer allows for sweeping of electrical current in the microfluidic cell.

Structures mimicking formation rock may be photolithographically patterned on the substrate when the proper mask is used. When this is done in such a method as method 300 or method 400, photolithographically patterning formation rock will yield a microfluidic cell that can be used to study aspects of formation rock properties, such as pore throat size when the inlet and outlet pressures are monitored. In one or more embodiments, connecting a current-voltage analyzer to the microfluidic cell allows for application of electrical current. In such embodiments the microfluidic cell can mimic and study the conditions of EEOR in a mature well's characteristic rock formation.

In another aspect, embodiments herein disclosed relate to a method of measuring pore throat size changes and oil mobilization. As shown in FIG. 5, method 500 includes connecting a current-voltage analyzer to a microfluidic cell, at block 501, and sweeping a current through the microfluidic cell via the current-voltage analyzer, at block 503. The microfluidic cell is one such as is found in the device of FIG. 1, or one made by the methods 300 or 400 as depicted in FIGS. 3 and 4 respectively. Patterning the substrate on formation rock produces a microfluidic cell that will mimic the formation rock behavior, allowing the device to study aspects of the formation. Pressures can be measured at the inlet and outlet and optionally the current-voltage analyzer may be used to apply current to the microfluidic cell. When the substrate mimics formation rock through photolithographic patterning and pressures are measured at the inlet and outlet during fluid pumping, the device will enable measurements of key formation properties such as pore throat size.

In one or more embodiments, connecting a current-voltage analyzer to the microfluidic cell at block 501 will allow for sweeping of electrical current in the microfluidic cell. In such embodiments, sweeping a current through the microfluidic cell via the current-voltage analyzer at block 503 includes gradually increasing the current with an increasing range of voltage, and this can then be used to probe the effect of electrical current on rock formation properties to model reservoir EEOR in a microfluidic device. When the current is swept, there may be resulting changes in the measured pore throat size according to pressure measurements at the inlet and outlet of the microfluidic cell. The current may be swept with an increasing range of voltage to cause a pore throat size increase. Thus, a maximum pore throat size increase can be observed. The observed maximum pore throat size increase provides a measure of enhanced oil mobilization at certain electrical conditions. Adjusting the current and voltage to find the maximum observed pore throat size may determine the ideal electrical conditions for EEOR, where the use of energy is most efficient for oil production. Further, the electrical conditions that result in an increased pore throat size and thus oil mobilization in the microfluidic device can be applied to the EEOR process in a mature well, in order to increase the formation pore throat size and thus oil mobilization. Accordingly, the measurements of increased pore throat size can be used to optimize the electrical conditions in EEOR. The optimization of the electrical conditions for EEOR can increase the production and reduce the costs of production in a mature well utilizing EEOR.

Embodiments of the present disclosure may provide at least one of the following advantages. Maximizing the measured pore throat size in EEOR can be useful for increasing efficiency and productivity of EEOR. This utilizes the ability of EEOR to be a tunable and adjustable method of EOR where the oil properties downhole can be adjusted by electrical conditions. Finding current-voltage conditions that yield optimized productivity in a well can improve the EEOR process by increasing production and/or reducing costs. Facile methods of determining the electrical conditions that lead to pore throat size increase such as disclosed herein can optimize EEOR.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed:

1. A device comprising:
a microfluidic cell comprising a substrate and metal electrodes disposed on the substrate;
a glass window disposed over the substrate;
a current-voltage analyzer connected to the metal electrodes; and
an inlet and an outlet in fluid communication with the microfluidic cell.

2. The device of claim 1, wherein the substrate comprises silica.

3. The device of claim 1, wherein the metal electrodes comprise gold.

4. A method of fabricating a device, the device comprising a microfluidic cell comprising a substrate and metal electrodes disposed on the substrate; a glass window disposed over the substrate; a current-voltage analyzer connected to the metal electrodes; and an inlet and an outlet in fluid communication with the microfluidic cell, the method comprising:
photolithographically exposing site for the metal electrodes utilizing a photoresist; depositing the metal electrodes on the substrate; and
photolithographically patterning the substrate.

5. The method of claim 4, wherein the depositing comprises chemical vapor deposition of the metal electrodes.

6. The method of claim 4, wherein the metal electrodes comprise gold electrodes.

7. The method of claim 4, wherein the photolithographically exposing site for the metal electrodes comprises coating the photoresist on the substrate.

8. The method of claim 4, wherein the photolithographically exposing site for the metal electrodes further comprises baking out the photoresist.

9. The method of claim 4, wherein the photolithographically patterning the substrate comprises etching the photoresist.

10. The method of claim 4, wherein the photolithographically patterning the substrate comprises etching the substrate.

11. The method of claim 4, wherein the photolithographically patterning the substrate comprises patterning a plurality of channels into the substrate.

12. The method of claim 4, wherein:
photolithographically exposing site for the metal electrodes comprises:
coating a photoresist on a substrate;
baking out the photoresist; and
removing portions of the photoresist with ultraviolet lithography to provide exposed portions of the substrate;
depositing the metal electrodes on the substrate comprises:
depositing metal electrodes on the exposed portions of the substrate; and
photolithographically patterning the substrate comprises:
exposing the photoresist and substrate to ultraviolet radiation with a mask to form a pattern in the photoresist;
dry etching the substrate to form a plurality of channels in the substrate defined by the pattern in the photoresist; and
removing the photoresist.

13. The method of claim 12, further comprising:
attaching a window to the substrate; and
connecting a current-voltage analyzer to the metal electrodes.

14. The method of claim 12, wherein the metal electrodes have been deposited using chemical vapor deposition.

15. A method of measuring pore throat size changes and oil mobilization including:
connecting a current-voltage analyzer to a microfluidic cell; and
sweeping a current through the microfluidic cell via the current-voltage analyzer.

16. The method of claim 15, wherein sweeping the current comprises gradually increasing the current with an increasing range of voltage.

17. The method of claim 16, wherein sweeping the current causes a measured pore throat size increase.

18. The method of claim 17, wherein the method comprises observing a maximum pore throat size increase.

19. The method of claim 18, wherein the maximum pore throat size increase provides a measure of enhanced oil mobilization.
